(19) United States
(12) Patent Application Publication
(10) Pub. No.: US 2015/0065456 A1
(43) Pub. Date: Mar. 5, 2015

(54) AGENT FOR TREATMENT AND PREVENTION OF CANCER

(71) Applicant: Daiichi Sankyo Company, Limited, Tokyo (JP)

(72) Inventor: Michele Mercuri, Scotch Plains, NJ (US)

(21) Appl. No.: 14/471,636

(22) Filed: Aug. 28, 2014

Related U.S. Application Data
(60) Provisional application No. 61/871,389, filed on Aug. 29, 2013.

(51) Int. Cl. A61K 31/444; A61K 45/06; A61N 5/10; A61K 31/727

(52) U.S. Cl. CPC A61K 31/444; A61K 31/727; A61K 45/06; A61N 5/10
USPC 514/56; 514/301

(57) ABSTRACT

The invention provides methods of treating or preventing in a subject a cancer, tumor, or neoplasm, including malignancies or metastases thereof, using a direct Factor Xa inhibitor. The methods particularly involve the treatment of human patients afflicted with a malignant cancer, tumor, or neoplasm with an effective amount of the Factor Xa inhibitor. In an embodiment, the Factor Xa inhibitor is the small molecule edoxaban p-toluenesulfonate monohydrate, also termed “DU-176b”. The invention provides methods of administering a Factor Xa inhibitor to effectively reduce or suppress, or otherwise abrogate, the growth of cancers, tumors, or neoplasms in a subject who has, or is at risk for, cancer and, optionally, who also has, or is at risk for, thrombotic disease or embolism. The methods of the invention can also reduce the incidence of migration or invasion of metastatic or malignant cancers in a human subject by administration of a Factor Xa inhibitor.

### FIELD OF THE INVENTION

The invention relates generally to treatment and prevention of cancers, particularly human cancers in patients also manifesting or at risk for thrombotic disease. The invention further generally relates to methods of inhibiting or reducing metastasis of various cancers.

### BACKGROUND OF THE INVENTION

Currently available methods of cancer therapy, e.g., surgical therapy, radiotherapy, chemotherapy, and immunotherapeutic or immunobiological procedures, have either limited success in preventing and treating cancers, including metastatic cancers, or give rise to serious and undesirable side effects. For a number of clinically-diagnosed solid tumors having localized growth, surgical removal is often considered the primary treatment choice. Unfortunately, in many cases following surgery, and frequently after a significant period of delay, the original tumor is observed to have metastasized, resulting in secondary sites of cancer invasion throughout the body; the patient subsequently dies of the secondary cancer growth. Reports indicate that in individuals with resectable tumors, primary tumor growth or local recurrence is not often the cause of death. Instead, at present, nearly 40% of cancer victims with operable tumors ultimately succumb to metastatic disease following surgery.

Although chemotherapy is widely used in the treatment of cancer, it is a systemic treatment, most often based on targeting the prevention of cell proliferation. As a result, chemotherapy is a non-specific treatment modality that affects virtually all proliferating cells, including normal cells, thereby leading to undesirable and often serious and deleterious side effects, such as immunosuppression, pancytopenia (growth inhibition of bone marrow cells with anemia, thrombocytopenia, and leukopenia), diarrhea, nausea and alopecia. Moreover, the existing systemic treatments have proven to have little effect on macro-metastases which already reside in remote organs, e.g. lung, liver, bone marrow, or brain. Patients succumb to metastatic cancers triggered by the metastasis of cancer cells.

Although a primary cancer may be completely eliminated, a malignant tumor will often be metastatic. The formation of metastases of malignant tumors, initiated from a primary tumor both proximal and distal locations in the body, is one of the most serious effects of cancer and one for which a wholly satisfactory treatment protocol is currently unavailable. Cancer tumor metastasis is responsible for most therapeutic failures when the disease is treated, largely because patients succumb to the multiple tumor growth. The extent to which metastasis occurs varies with the individual type of tumor. Melanoma and cancers of the breast, lung, colon and prostate are examples of types of cancers that are prone to metastasize. Metastases can form at a variety of sites in the body, with lymph nodes, lungs, liver, brain and bone marrow being the more common sites.

Methods for effectively reducing or suppressing the metastasis of cancer cells have not been established, nor has a therapeutic drug or medicine having a cancer cell metastasis reducing or suppressing effect yet become commercially available. Accordingly, there exists a need for methods of reducing and/or inhibiting tumor metastasis. Of particular importance are methods that reduce or inhibit metastasis without causing adverse or serious side effects. In addition, cancer may be associated with increased coagulation and risk of thrombosis and, in fact, thrombosis is a common cause of mortality in cancer patients. Accordingly, there is a need for anti-cancer agents that also treat, prevent or reduce the risk of thrombosis. Novel and effective agents and methods for reducing cancer metastases would greatly aid the medical community and patients, since the need is great. The present invention provides a means to address and fulfill such a need.

### SUMMARY OF THE INVENTION

It is an aspect of the invention to provide methods of treating or preventing a cancer, tumor, or neoplasm, including malignancies or metastases thereof, in a subject by administration of a Factor Xa inhibitor. In aspects of the invention, the subject is a human patient suffering from cancer; a patient at risk of cancer due to genetic predisposition, or environmental exposure and the like; a patient in whom a cancer has recurred; or a patient in remission from cancer; or a patient at risk for recurrence of cancer. The methods particularly involve the treatment of human patients afflicted with a malignant cancer, tumor, or neoplasm with an effective amount of the Factor Xa inhibitor. Further, the methods of the invention involve treating or preventing cancer, particularly, malignancy and metastasis, by administration of a Factor Xa inhibitor in a patient that also has a thrombotic disease or is at risk of developing thrombotic disease. In an aspect, the Factor Xa inhibitor is a direct Factor Xa inhibitor. In an aspect, the Factor Xa inhibitor is the small molecule edoxaban, or a pharmaceutically acceptable salt and/or hydrate thereof. In an aspect, the Factor Xa inhibitor is edoxaban p-toluenesulfonate monohydrate (termed “DU-176b” herein and also referred to as edoxaban tosylate). In an aspect, edoxaban is the anhydrous free base of edoxaban tosylate.

In an aspect, the invention provides a method of treating or preventing a cancer or neoplasm in a human subject in need thereof, in which the method involves administering to the subject a pharmaceutical composition comprising a direct Factor Xa inhibitor in a therapeutically effective amount to said human subject, thereby treating or preventing the cancer or neoplasm. In an aspect of the invention, the direct Factor Xa inhibitor is administered in solid form, such as a tablet, pill, capsule, and the like. In an aspect of the invention, the direct Factor Xa inhibitor is orally bioavailable and is orally administered to a subject in need thereof. In a specific aspect, the Factor Xa inhibitor is in a solid dosage form.

In an aspect, the invention provides a method of treating a cancer or neoplasm in a human subject, in which the method involves administering to the subject a pharmacologically effective amount of heparin; subsequently discontinuing heparin administration to the subject; and administering a pharmaceutical composition comprising a direct Factor Xa inhibitor in a pharmacologically effective amount to the subject, thereby treating the cancer or neoplasm in the subject. In an aspect of the method, the administration of heparin is discontinued after five days or after about five days. In aspects of the method, the direct Factor Xa inhibitor is in solid dosage form and/or is orally administered.

In an aspect, the invention provides a method of reducing metastasis of a cancer or neoplasm in a human subject in need thereof, in which the method involves orally administering to the subject an effective amount of a direct inhibitor of Factor Xa. In an aspect, the Factor Xa inhibitor is administered to a human patient in need of treatment for thrombosis and who has cancer or who is at risk for cancer, including metastatic and malignant cancer. In an embodiment, the Factor Xa inhibitor is administered to a human patient in need of treatment or prevention of cancer in that the human patient has cancer or is at risk for or predisposed to cancer and is also at increased risk for thrombosis as compared to the general population.

In an aspect related to the above methods, the direct Factor Xa inhibitor is N¹-(5-chloropyridin-2-yl)-N²-(1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide having the structure:

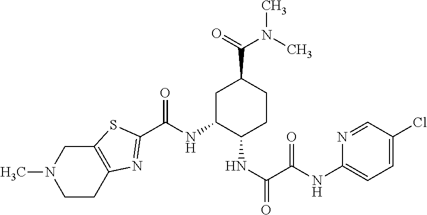 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

or a pharmaceutically acceptable salt and/or hydrate thereof.

In a particular aspect, the direct Factor Xa inhibitor is the p-toluenesulfonate salt of N¹-(5-chloropyridin-2-yl)-N²-(1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide.

In a more specific aspect, the direct Factor Xa inhibitor is N¹-(5-chloropyridin-2-yl)-N²-(1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide p-toluenesulfonate monohydrate, having the formula below:

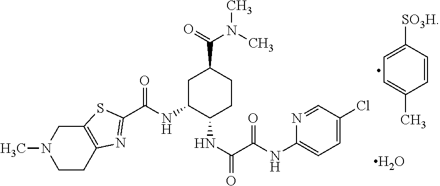

In accordance with the invention, edoxaban (INN) refers to the anhydrous free base of edoxaban tosylate (DU-176b). In an aspect, human subjects are administered edoxaban tosylate (a monohydrate salt form), but all doses and plasma concentrations are expressed in terms of edoxaban (the anhydrous free base of edoxaban tosylate).

In another of its aspects, the invention provides a method of reducing or preventing metastasis of a cancer or neoplasm in a patient, preferably a human patient having or being at risk for thrombotic disease, which method comprises treating the patient with an amount of edoxaban effective to reduce or prevent metastasis and to reduce or prevent thrombotic disease. In specific embodiments, the methods of the invention comprise administration to a human patient having or being at risk for thrombotic disease and having or being at risk for cancer of a therapeutically effective amount of a Factor Xa inhibitor which results in the treatment and/or prevention of the thrombotic disease and the cancer but also does not result in an increased bleeding risk or a bleeding risk that outweighs the benefit of the anti-thrombotic/anti-cancer effect of the Factor Xa inhibitor.

In an aspect of the above methods, the dosage of edoxaban (as the free base) administered is from 0.1 mg to at least 90 mg per day; or from 5 mg to 90 mg per day; or from 30 mg to 60 mg per day. In an aspect of the above methods, the effective amount of edoxaban is 60 mg per day. In another aspect of the above methods, the effective amount of edoxaban is 30 mg per day. In another aspect of the above methods, the administering comprises oral administration. In another aspect of the above methods, the cancer or neoplasm is selected from a solid tumor or neoplasm, a non-solid tumor, a lymphoma, or a leukemia. In another aspect, the cancer or neoplasm is located in or on a body tissue or organ selected from thyroid, lung, stomach, small bowel, large bowel, liver, kidney, pancreas, prostate, uterus, genital, or bladder. In another aspect of the methods of the invention, the FXa inhibitor, including edoxaban, exhibits significant anticoagulant and antithrombotic effects in the subject undergoing treatment. In other aspects, the methods further comprise treating the subject with an additional chemotherapeutic agent. In some aspects of the methods, the chemotherapy agent is selected from the group consisting of an alkylating agent, an anti-metabolite, a mitotic inhibitor, a cytotoxic antibiotic, a compound that damages DNA and a compound that interferes with DNA expression. In another aspect of the invention, the methods further include surgical intervention and/or treating the subject with radiotherapy.

The foregoing and other aspects, features and advantages of the invention and its embodiments will become apparent in the descriptions of the accompanying drawings and embodiments provided herein.

### BRIEF DESCRIPTION OF THE DRAWINGS

FIG. 1 shows a plot of Kaplan-Meier cumulative rate estimates for investigator-reported malignancies that were clinically evident post-randomization in a treatment +30 days study period in connection with the Hokusai VTE clinical study. The treatment groups are indicated as follows: solid line (Edoxaban); broken line (Warfarin). Below the x-axis are the numbers of subjects on the edoxaban or warfarin study drugs corresponding to days from initial study drug administration. Over a year, e.g., 390 days, from initial study drug administration, the edoxaban-treated subjects having a history of prior malignancies showed a significant decrease in the incidence (%) of new or recurrent malignancies versus the warfarin-treated subjects.

FIG. 2 shows a plot of Kaplan-Meier cumulative rate estimates for investigator-reported malignancies that were clinically evident post-randomization in the overall study period in connection with the Hokusai VTE clinical study. The treatment groups are indicated as follows: solid line (Edoxaban); broken line (Warfarin). At about 360 days from initial study drug administration, the edoxaban-treated subjects having malignancies showed a significant decrease in the incidence (%) of new or recurrent malignancies versus the warfarin-treated subjects.

### DETAILED DESCRIPTION

The invention provides a method of treating, preventing or reducing the incidence of a cancer, e.g., a tumor or neoplasm, in particular, a malignant or metastatic cancer, tumor, or neoplasm, with an inhibitor of Factor Xa (FXa). In an embodiment, the cancer is a malignant or metastatic cancer, tumor, or neoplasm, and the FXa inhibitor is a small molecule inhibitor of the FXa serine protease. In an embodiment, the FXa inhibitor is a direct inhibitor of FXa activity. In an embodiment, the FXa inhibitor is an indirect inhibitor of FXa activity, e.g., by interacting with prothrombin. In an embodiment, the FXa inhibitor is a direct inhibitor of FXa activity that exhibits both FXa inhibiting effects and has antithrombotic, as well as anticoagulant effects.

FXa, a key serine protease coagulation factor, plays a vital role in thrombosis and hemostasis by enzymatically cleaving its substrate, prothrombin, to produce thrombin in the coagulation cascade, thereby regulating the generation of this important procoagulant enzyme. In addition, FXa contributes to additional physiological and pathophysiological mechanisms by eliciting a number of critical cellular responses, such as cytokine release, adhesion molecule expression, tissue factor (TF) expression, and cell proliferation, which are mediated by specific receptors and are involved with intracellular and/or extracellular signaling molecules and mediators. The binding of FXa to cells and the resulting stimulation or activation of cellular events positions FXa as a contributing force in a variety of diseases and pathologies, including cancer. Regardless of the disease, the inhibition of FXa in the coagulation cascade prolongs clotting time and potentially reduces the risk of spontaneous or induced thrombus formation, thereby having a beneficial effect on patient treatment.

Deep vein thrombosis (DVT) and pulmonary embolism (PE) are considered to be manifestations of a single pathophysiologic process that is collectively known as venous thromboembolism (VTE). DVT and PE frequently present together, share the same risk factors and are associated with a high morbidity that may progress to a fatal outcome, if left untreated. VTE is a common disorder with an estimated annual incidence of approximately 2-3 per thousand individuals. While DVT is a blood clot found anywhere in the deep veins of the legs, pelvis or arms, PE occurs when part of a clot from within a deep vein detaches and embolises to the lungs, lodging in the pulmonary arteries and causing a potentially fatal condition. Of additional concern, recent National Institute for Health and Care Excellence (NICE) guidelines have highlighted the fact that for individuals suffering from DVT, the condition might indicate the presence of an undiagnosed cancer or malignancy.

Abnormal hemostasis, including DVT, is a fundamental property of malignant disease, and is not merely a secondary phenomenon attributable to therapy or to chronic illness. Almost anyone who is fighting cancer is at risk for developing DVT; indeed, more than 90 percent of patients with cancer may be at risk for DVT. Complications from DVT are the second leading cause of death among cancer patients. Additionally, more than 50 percent of people who died from certain cancers had developed a DVT. The procoagulant activities of some malignancies, including cancers, tumors and neoplasms, can lead to further pathological conditions for patients afflicted with such malignancies, including disseminated intravasular coagulation (DIC). Having cancer may increase one's risk for blood clots because surgery or chemotherapy may injure blood vessel walls, causing coagulation, and certain types of cancer treatments may reduce the body's ability to produce natural anticoagulant agents. The risk for developing DVT may be increased for those individuals having cancer of the ovaries, pancreas, lymphatic system, liver, stomach and colon, without limitation. In addition, undergoing chemotherapy may increase the likeliness of DVT, thus, placing cancer patients at even higher risk while undergoing this treatment. For those receiving chemotherapy treatment, the risk of DVT can be twice as high compared with those who are cancer-free.

Many types of tumor cells express clotting initiators such as tissue factor (TF), and are also involved at a later stage in the coagulation pathway by providing a surface for prothrombinase generation. Thus, entry of tumor cells into the plasma, as during metastasis, may be expected to trigger intravascular clotting. However, and importantly, solid tumors growing outside of the blood vasculature regularly deposit fibrin locally in the tissues by rendering the microvasculature hyperpermeable. This allows fibrinogen and other plasma-clotting proteins to leak into the extravascular space where procoagulants associated with tumor cells or with benign stromal cells initiate clotting. Both fibrin deposition and turnover in solid tumors proceed at rapid rates and may occur due to events in the intra- or extra-vascular space. The abnormal clotting and fibrinolysis can override normal regulatory mechanisms, resulting in a lack of protection from host inflammatory cells, modulation of the immune response, and induction of angiogenesis.

In an embodiment, the invention provides an inhibitor of FXa for use in the reduction or suppression of cancer, including the reduction, suppression, or prevention of metastases, in human subjects. In an embodiment, the invention provides an inhibitor of FXa for use in the reduction, suppression, or prevention of the incidence of new or recurrent metastases in human subjects. In an embodiment of the invention, the FXa inhibitor exhibits anti-thrombotic and anticoagulant activities and also modulates and prevents the incidence of metastasis of a number of different cancer types. In accordance with the invention, such an FXa inhibitor is employed as an anticancer drug in methods of treating humans having cancer, including metastatic cancer, as well as in treating humans afflicted with both cancer and thrombosis-related diseases (or at risk for thrombosis-related diseases), such that the inhibitor is dually effective in reducing, suppressing, or preventing the spread or progression of metastatic cancer, and in its activity as an anticoagulant and antithrombotic agent, in the absence of significant, accompanying adverse effects, such as bleeding, in the subjects undergoing treatment. The FXa inhibitor may also be used to prevent or reduce the incidence of cancer in a subject at risk for or having a predisposition for cancer or metastatic cancer and also to treat or prevent thrombosis in a patient having thrombotic disease or at risk for or having a predisposition for thrombotic disease.

In an embodiment, the invention contemplates methods of treating cancer, resulting in the reduction, suppression, or prevention of cancers, including the spread of metastases, in human subjects, in which the FXa inhibitor can be selected from direct (e.g., direct binding to FXa) or indirect (e.g. activity dependent upon antithrombin) inhibitors of FXa. In an embodiment, the direct FXa inhibitor is orally active. Nonlimiting examples of direct FXa inhibitors include rivaroxaban (Bayer Healthcare AG and Scios, Inc.), otamixaban (Sanofi-Aventis), LY517717 (Lilly), YM150 (Astellas), apixaban and razaxaban (Bristol-Myers Squibb), 813893 (GlaxoSmithKline), PRT054021 (MLN-1021), (Portola), AVE-3247, EMD-503982, or KFA-1982. Non-limiting examples of indirect FXa inhibitors include low molecular weight (LMW) heparins, low molecular weight lignins (LMWLs), fondaparinux (Arixtra®) (GlaxoSmithKline), and Idraparinux sodium (Sanofi-Aventis and Organon). In a particular embodiment, the Factor Xa inhibitor used in the methods of the invention is not the 3-amidoinophenylalanine-type FXa inhibitor called WX-FX4, which shows weaker anticoagulant activity compared to other FXa inhibitors. In other embodiments, the FXa inhibitor has more potent anti-coagulant activity than WX-FX4. For example, while WX-FX4 exhibits an inhibition constant (Ki) value for human FXa of 74 nM, edoxaban p-toluenesulfonate monohydrate (DU-176b) exhibits a Ki value of 0.561 nM. As another example, DX-9065a, a non-orally available inhibitor of FXa, exhibits a Ki value of 41 nM. In a particular embodiment, the FXa inhibitor used in the method of the invention is not DX-9065a.

In a particular embodiment, the invention provides a method of treating a cancer, particularly a malignant or metastatic cancer, in which the FXa inhibitor is edoxaban p-toluenesulfonate monohydrate (also called “DU-176b” herein). Edoxaban p-toluenesulfonate monohydrate is a potent, orally active, selective, direct and reversible inhibitor of FXa, manufactured by Daiichi Sankyo Co., Ltd., Japan. (See, e.g., T. Furugohri et al., 2008, “D-176b, a potent and orally active factor Xa inhibitor: in vitro and in vivo pharmacological profiles”, J. Thrombosis and Haemostasis, 6:1542-49; and U.S. Pat. No. 7,365,205, the contents of which are incorporated by reference herein in their entirety).

In a particular embodiment, the direct Factor Xa inhibitor is N¹-(5-chloropyridin-2-yl)-N²-4-[(dimethylamino)carbonyl]-2-{[(5-methyl-4,5,6,7-tetrahydrothiazolo[5,4-c]pyridin-2-yl)carbonyl]amino}cyclohexyl)ethanediamide having the structure:

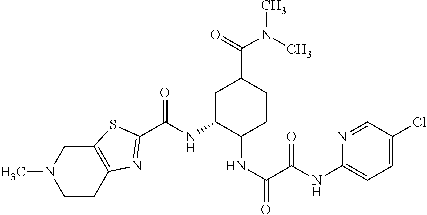 [SMILES: CN1CCc2nc(C(=O)N[C@@H]3CC(C(=O)N(C)C)CCC3NC(=O)C(=O)Nc3ccc(Cl)cn3)sc2C1]

The Factor Xa inhibitor may be a pharmaceutically acceptable salt and/or hydrate of N¹-(5-chloropyridin-2-yl)-N²-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, such as a p-toluenesulfonate salt, and/or a hydrate thereof, particularly a monohydrate. Other pharmaceutically acceptable salts include conventional, relatively non-toxic, inorganic or organic addition salts or quaternary ammonium salts of the parent compound formed, for example, from non-toxic inorganic or organic acids. For example, such conventional non-toxic salts include those derived from inorganic acids such as hydrochloric acid, hydrobromic acid, sulfuric acid, sulfamic acid, phosphoric acid, nitric acid and the like; and those prepared from organic acids such as acetic acid, propionic acid, succinic acid, glycolic acid, stearic acid, lactic acid, maleic acid, tartaric acid, citric acid, ascorbic acid, pamoic acid, maleic acid, hydroxymaleic acid, phenylacetic acid, glutamic acid, benzoic acid, valeric acid, oleic acid, salicylic acid, sulfanilic acid, 2-acetoxybenzoic acid, fumaric acid, p-toluenesulfonic acid, methanesulfonic acid, ethane disulfonic acid, oxalic acid, ethylenediaminetetraacetic acid, formic acid, benzene sulfonic acid, naphthalene-2-sulfonic acid, 3-hydroxy-2-naphthalenecarboxylic acid, and the like. (See, also, e.g., S. M. Barge et al. 1977, Pharmaceutical Salts, J. Pharm. Sci., 66:1-19). Such physiologically acceptable salts are prepared by methods known in the art, e.g., by dissolving the free amine bases with an excess of the acid in aqueous alcohol, or by neutralizing a free carboxylic acid with an alkali metal base, such as a hydroxide, or with an amine.

In an embodiment, the methods of the invention embrace the use of stereoisomers of N¹-(5-chloropyridin-2-yl)-N²-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, in particular N¹-(5-chloropyridin-2-yl)-N²(1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, which has the structure reproduced below.

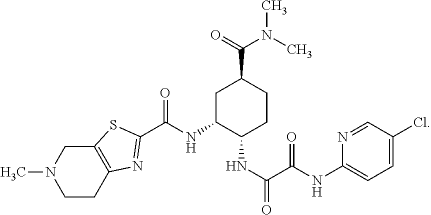 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

The Factor Xa inhibitor may also include pharmaceutically acceptable salts and/or hydrates of N¹-(5-chloropyridin-2-yl)-N²(1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, particularly, p-toluenesulfonate salts and either the anhydrous or the monohydrate forms.

In a specific embodiment, Factor Xa inhibitor is edoxaban p-toluenesulfonate monohydrate, which has the formula: N¹-(5-chloropyridin-2-yl)-N²-(1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide p-toluenesulfonate monohydrate and the structure:

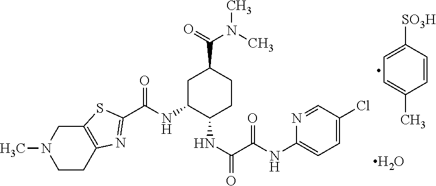

Edoxaban is orally bioavailable, as demonstrated in preclinical pharmacodynamic/pharmacokinetic (PD/PK) studies in rats and monkeys, as well as in clinical studies in human subjects. Edoxaban has been shown to be generally safe and well tolerated in doses of up to 90-mg per day. Moreover, in its capacity as an antithrombotic agent and anticoagulant, edoxaban p-toluenesulfonate monohydrate (DU-176b) potently inhibits both free FXa and FXa complexed in prothrombinase with a subnanomolar Ki value, and its inhibitory activity is highly specific. For example, DU-176b exhibits a >10.000-fold more potent inhibition of FXa than other biologically relevant serine proteases.

In accordance with the invention, human subjects with cancer malignancies or metastases, who were in need of anti-thrombotic therapy and who were administered edoxaban p-toluenesulfonate monohydrate (as the Active Pharmaceutical Ingredient (API)) in a clinical study as described herein, showed, upon an evaluation of clinical data following edoxaban treatment, a surprising and unexpected reduction in the incidence of new or recurrent malignancies or metastases, as compared to the study subjects who were administered warfarin. The study was a randomized, double-blind, clinical study, in which subjects with symptomatic venous thromboembolism (VTE), which included PE, with or without deep venous thrombosis (DVT), or DVT only, were treated with either edoxaban or warfarin, a Vitamin K antagonist, which is an indirect inhibitor of coagulation. The purpose of the clinical study was to evaluate whether an initial regimen of enoxaparan (i.e., low molecular weight (LMW) heparin) followed by edoxaban is non-inferior to a regimen of enoxaparan followed by warfarin for the treatment and long term prevention of recurrent, symptomatic VTE, defined as DVT in a proximal leg vein (popliteal or above), pulmonary embolism (PE), or VTE-related death. The subjects participating in the study were adult male and female patients presenting with acute, symptomatic DVT involving popliteal, femoral, or ileac veins, or PE requiring anti-thrombotic therapy. Patients with various types of cancer were among the subjects who were treated in this study, so long as long term treatment with heparin was not anticipated as a treatment regimen for such patients. This clinical study is named Hokusai-VTE and is the largest single phase 3 clinical study in the treatment and prevention of recurrence of VTE using an FXa inhibitor such as edoxaban. More than 8,250 patients were enrolled in the study from more than 400 clinical sites across 38 countries worldwide. The Hokusai-VTE clinical study was designed to reflect clinical practice, using a standard heparin lead-in and providing a flexible treatment duration of three, six or twelve months. This study design allowed the evaluation of patients with a broad range of risks, including patients with moderate or severe conditions of PE and DVT. In addition, the study did not exclude subjects with cancer, including malignancies. (See, Example 1, infra).

In evaluating the study treatment results at different time periods for information relating to the potential incidence of new cancers in subjects in the edoxaban treatment program, a statistically significant reduction in the incidence of clinically-evident, investigator-reported malignancies was surprisingly observed in edoxaban-treated subjects enrolled in the study, compared with subjects in the non-edoxaban, i.e., warfarin, treatment arm. The reduction in incidence of newly observed malignancies of different cancer types was found to range from 16% to 27%. In an example, the cancer types affected are solid tumors or neoplasms. In another example, the cancer types affected are leukemias or blood-related cancers. In another example, the cancers are malignant or metastatic. In another example, the cancers are malignant or metastatic solid tumors or neoplasms. Thus, the invention provides a novel cancer treatment, either via preventative or therapeutic avenues, to treat cancers of various types, and/or to inhibit or reduce the progression or spreading of cancer cells through the metastatic process, by means of treating a subject with an effective amount of the FXa inhibitor, e.g., edoxaban.

In embodiments of the invention, subjects undergoing treatment with the FXa inhibitor can be symptomatic or asymptomatic for a number of conditions related to thrombotic disease or pathology. In particular, the subject, preferably human subjects, have or are at risk for thrombotic disease in addition to having or being at risk for cancer, particularly malignant cancer and metastasis. In certain embodiments, by virtue of being afflicted with cancer and/or subject to treatment for cancer, the patient is at increased risk for developing a thrombotic condition as compared to a subject that does not have cancer. In other embodiments, the subject has a thrombotic condition and also has cancer or is at risk for cancer, or has cancer that is at risk for metastasis. Such patients may have or be at risk for thrombotic conditions that include, without limitation, VTE, DVT, PE, embolism, thromboembolism, and venous thrombosis. The thrombotic conditions also include peripheral arterial disease, atrial fibrillation, thrombotic events following surgery, for example but not limited to, hip replacement, knee replacement, or other orthopedic surgery. In addition, in the methods of the invention the subjects to be treated with a Factor Xa inhibitor such as edoxaban may be afflicted with, or susceptible to, cerebral infarction, cerebral embolism, myocardial infarction, angina pectoris, pulmonary infarction, pulmonary embolism, Buerger's disease, deep venous thrombosis, disseminated intravascular coagulation syndrome, thrombus formation after valve or joint replacement, thrombus formation and reocclusion after angioplasty, systemic inflammatory response syndrome (SIRS), multiple organ dysfunction syndrome (MODS), thrombus formation during extracorporeal circulation, or blood clotting upon blood drawing. In an embodiment, the VTE may encompass PE with or without DVT or DVT only. (See, Example 1, infra).

Accordingly, the invention provides a novel treatment regimen for cancer therapy and for reducing malignancies, and the incidence of metastases, in individuals afflicted with cancer and metastatic cancer. Further, the invention provides advantages and advances in the treatment of cancer, particularly metastatic cancer, in that edoxaban, as an inhibitor of FXa activity, is not only effective in treating thrombosis and coagulation disorders, but it also provides a novel and effective treatment for reducing cancers and tumors, including metastatic progression of cancer. In an embodiment, the subject who is afflicted with cancer may also have associated venous thromboembolism disease, i.e., VTE, including DVT and PE, or another thrombotic disease as described supra, which is also effectively treated by edoxaban administration, thereby providing a treatment both for inhibiting the migration or progression of metastatic cancers and for treating thrombosis and embolisms, e.g., VTE, in those subjects afflicted with both thrombotic diseases and cancer (metastases). In an embodiment, the invention provides a method of suppressing metastasis in those patients suffering from both cancer, metastatic and malignant cancer, and thrombosis-related diseases, using a FXa inhibitor such as edoxaban, wherein the method does not lead to excessive levels of bleeding or other adverse effects. In an embodiment, the invention provides methods of treatment in which an FXa inhibitor such as edoxaban suppresses metastasis and provides a prolongation of survival in subjects, preferably human patients, suffering from cancer and/or metastasis.

The term “cancer” as used herein is understood to encompass neoplasms and tumors, which refer to abnormal growths or abnormally growing cells that can invade surrounding tissues and spread to other organs, i.e., become malignant, if left untreated. Neoplasms are abnormal growths of tissue that form as a result of neoplasia, which is the abnormal growth and proliferation of cells, either malignant or benign; neoplasms include the abnormal growths of precancerous and cancerous cells and tissues. Neoplasms and tumors (which are types of neoplasms) refer to abnormal growths or masses of tissues comprised of cells, whether precancerous or cancerous, that grow more rapidly than normal cells and that will continue to grow and compete with normal cells for nutrients if not treated. Neoplasms may include, without limitation, solid and non-solid, such as hollow or liquid-filled, tumors and also hematological cell neoplasias or neoplasms, e.g., lymphomas, leukemias and myelomas. The terms metastasic cancer and malignant cancer are used interchangeably herein. Metastasis or metastatic disease refers to the spread of a cancer from one tissue, organ, or body part to another non-adjacent tissue, organ, or body part. The new occurrences of disease thus generated and spread are referred to as metastases or “mets”. In addition, the term ‘cancer’ as used herein is intended to embrace neoplasms and tumors of various origins within and on the body, types and subtypes, as well as organ, tissue and cell samples and specimens, e.g., biological samples or specimens thereof.

Neoplasms include all forms of cancer cells and cell masses known as tumors, which may be malignant or benign, and may be invasive or non-invasive. Tumors include solid tumors and disseminated tumors. For example, disseminated tumors include lymphomas and leukemias, and the like. As other nonlimiting examples, tumors, such as solid tumors, include adenocarcinomas, carcinomas, myelomas, melanomas, gliomas, sarcomas, adenosarcomas, adenomas and the like. Tumors can occur in virtually all parts of the human body, including every tissue and organ. The tumors may, for example, be present in the breast, heart, lung, small intestine, stomach, small bowel, large bowel, colon, spleen, kidney, liver, gall bladder, bile duct, bladder, head and neck, esophagus, thyroid, ovary, uterus, cervix, testicles, prostate, brain, pancreas, skin, bone, bone marrow, blood (leukemia), and thymus. These examples are included for illustrative purposes and are not intended to be limiting in any way. In an embodiment, the methods of the invention are particularly effective in reducing the incidence of solid tumors or neoplasms. In an embodiment, the methods of the invention involving edoxaban treatment are particularly effective in reducing the incidence of metastatic tumors or neoplasms selected from thyroid, lung, stomach, small bowel, large bowel, liver, gall bladder, bile duct, renal, prostate, bladder, genital tumors or neoplasms, and leukemias. In a particular embodiment, methods of treating or preventing solid tumors or neoplasms, are encompassed by the invention. In a particular embodiment, methods of treating, preventing or reducing the incidence of metastases of solid tumors or neoplasms, are encompassed by the invention.

In embodiments of the invention, the treatment methods involving the administration of an FXa inhibitor, such as edoxaban (administered API is edoxaban p-toluenesulfonate monohydrate) are suitable for human subjects who represent several different populations of cancer patients. Illustratively and without limitation, cancer patients, including those with metastatic or malignant cancer, can be those who currently have cancer; those who are in remission from cancer, those who are undergoing treatment for a cancer, those who may be at risk for developing a cancer, such as those having a family history of a particular form or type of cancer or a genetic predisposition; and those who have a recurrent cancer condition. In particular embodiments, the cancer patients may have tumors or neoplasms in which metastasis has not yet occurred, and the methods of the invention involve administration of the FXa inhibitor to prevent metastasis, e.g., to prevent or inhibit the migration and/or invasion of the tumor cells into distal tissues of the patient. In all of the foregoing patient populations, the cancer patients can also be in need of anti-thrombotic or anticoagulant treatment involving the use of an inhibitor of FXa, such as edoxaban. In an embodiment, the methods of the invention are suitable for a patient population including those individuals who are in need of anti-thrombotic treatment who are also at risk for cancer, including metastatic or malignant cancer. In other embodiments, the methods of the invention are suitable for a patient population in which the patient is in need of an anti-cancer treatment and is also at risk for or has thrombotic disease.

Without wishing to be limiting, treating a cancer, tumor, or neoplasm can involve the reduction of the formation or progression of a tumor or neoplasm by the practice of the invention, including reducing, diminishing, inhibiting, abrogating, eliminating, or ablating the proliferation or metastasis of the tumor or neoplasm. In addition, in an embodiment, the methods of the invention can prevent the initiation or establishment of the tumor or neoplasm, and/or can inhibit or moderate the proliferation or metastasis of the tumor or neoplasm. In another embodiment, the methods of the invention can reduce the incidence of, or inhibit the migration of, metastases and/or malignant cancers, tumors, or neoplasms.

In various embodiments, the responsiveness of a cancer to edoxaban treatment can be assessed using any endpoint indicating a benefit to the subject, including, without limitation (i) an extent of inhibition of cancer, tumor, or neoplasm growth, including growth rate reduction, reduction in progression, and complete growth arrest; (ii) reduction in the number of cancer, tumor, or neoplasm cells; (iii) reduction in cancer, tumor, or neoplasm size or volume; (iv) inhibition, e.g., reduction, lessening, or complete cessation of cancer, tumor, or neoplasm cell infiltration into adjacent peripheral organs and/or tissues; (v) inhibition, e.g., reduction, lessening, or complete cessation, of metastasis, or in the number of metastases, or in the incidence of metastases; (vi) enhancement of an anti-cancer, tumor, or neoplasm immune response, resulting, optimally, in the regression or rejection of the cancer, tumor, or neoplasm; (vii) relief, to some extent, of one or more symptoms associated with the cancer, tumor, or neoplasm; (viii) increase in the duration of survival/length of survival time following treatment; and/or (ix) decreased mortality subsequent to commencing and/or maintaining treatment.

The phrase “effective amount” refers to an amount or dose sufficient to effect a desired response, to reduce the incidence of, to prevent, or to ameliorate a symptom or sign, e.g., of metastasis or primary or secondary tumor progression, size, or growth. While human subjects and human patients are preferred, the invention contemplates the treatment of other, typical mammalian subjects, such as, for example, mice, rats, cats, dogs, horses, sheep, cows, and non-human primates. An effective amount for a particular patient may vary depending on factors such as the condition being treated, the overall health of the patient, the method, route, and dose of administration and the severity of side effects. Preferably, the effect will result in a change in quantification of at least about 10%, preferably at least 15%, 16%, 20%, 25%, 27%, 30%, 40%, 50%, 60%, 70%, 80%, 90%, or even 95% or more. Percentages of tumor reduction having values within or between the aforementioned percentages are also embraced by the invention. When in combination, an effective amount is in ratio to a combination of components and the effect is not limited to individual components alone. In an aspect, an effective amount of a therapeutic will modulate the symptoms typically by at least about 10% or more; or by at least about 20% or more; or by at least about 30% or more; or preferably by at least about 50% or more, such as 60%, 70%, 80%, or 90% or more. Alternatively, the modulation of migration will mean that the migration, spreading, or trafficking of various cell types, e.g., cancer cell types, is inhibited, suppressed, or reduced. This can result in, for example, statistically significant and quantifiable changes in the numbers of cancer cells whose migration or progression is inhibited, blocked, suppressed, or reduced. Rate of primary or secondary tumor progression, size, or growth can also be monitored.

Preferred doses and unit dosage formulations for FXa inhibitors, for example, direct FXa inhibitors, e.g., edoxaban, are those containing an effective dose, such as provided herein for guidance, or an appropriate fraction thereof, of the active ingredient. Factor Xa inhibitors that are small molecules may be administered at a dose of from 0.1 to 500 mg/kg per day. In various embodiments, the dose may be administered daily, twice a day, three times a day, every other day, every week, twice a week, every two weeks, every three weeks, etc., as will be appreciated by the skilled practitioner. Oral administration is preferred. The dose range for adult humans is generally from 5 mg to 2 g/day. Doses of FXa inhibitors may be administered as frequently or infrequently as determined by a patient's physician or medical doctor, and may be administered for a short time period, e.g., weeks, months, or for a longer time period, such as chronic administration, e.g., over several months or years. Tablets or other forms of presentation provided in discrete units may conveniently contain an amount of one or more compounds which is pharmaceutically and therapeutically effective at such dosage or as a multiple of the same, for instance, units containing 5 mg to 500 mg, usually around 10 mg to 200 mg, including discrete amounts there between, e.g., 10 mg, 20 mg, 30 mg, 40 mg, 50 mg, 60 mg, 70 mg, etc. In an embodiment, the dose is a solid dosage form. In an embodiment, the FXa inhibitor, e.g., edoxaban, is administered with food. In an embodiment, the FXa inhibitor, e.g., edoxaban, is administered without food.

In a specific embodiment, the Factor Xa inhibitor is edoxaban, and a therapeutic or effective amount of edoxaban for treatment or prevention of a cancer, or a metastatic cancer, is a dose which does not cause or result in an increased rate of bleeding following administration/dosing. In an embodiment, the effective amount of edoxaban for administration (wherein the administered API is edoxaban p-toluenesulfonate monohydrate) refers to the free base form or equivalent thereof, in which “equivalent thereof” means the same molar amount of the edoxaban free base active moiety regardless of the actual form administered. Accordingly, edoxaban (with dosage amounts being for the active moiety, the free base, and including the equivalent amount (e.g., the same molar amount of the free base) of any salt or hydrate or any other form thereof), may be administered in doses from 0.1 mg to at least 90 mg per day; or from 5 mg to 90 mg per day; or from 30 mg to 60 mg per day; or from 20 mg to 40 mg per day; or from 40 mg to 60 mg per day; or from 60 mg to 80 mg per day; or from 25 mg to 65 mg per day. The dose is preferably given once per day, but may also be given in multiple doses per day, for example, once, twice, three times, or four times a day. Alternatively, the dose may be given every other day or every three days, four days, or five days. Doses between the specified amounts in the ranges are also contemplated. In an embodiment of the inventive methods, the effective amount of edoxaban (as the free base) is 60 mg, or about 60 mg. In another aspect of the above methods, the effective amount of edoxaban (as the free base) is 30 mg or about 30 mg. In an embodiment, the dose is 60 mg, or about 60 mg, per subject once per day (QD). In another embodiment, the effective amount of edoxaban for treatment of a cancer, or a metastatic cancer, is 30 mg, or about 30 mg, once per day (QD). In an embodiment, for subjects considered as fragile (e.g., the elderly; those who are health-compromised in some way, e.g., moderate renal impairment; those who are receiving drug treatments for other conditions and pathologies, e.g., those receiving P-glycoprotein inhibitors; and those who develop tumors or cancer while undergoing treatment for VTE), edoxaban (as the free base) may be administered at a dose less than 60 mg, such as 30 mg, once daily.

In accordance with the invention, the FXa inhibitor can be administered by any route conventionally used for drug administration and as known to the skilled practitioner. By way of non-limiting example, administration may be oral, parenteral, intravenous, subcutaneous, bucal, sublabial, intranasal, intradermal, sublingual, intrathecal, intramuscular, intraperitoneal, rectal, intravaginal, gastric, or enteric. Oral administration, e.g., in single dosage form, solid dosage form, such as a tablet, or in liquid form, is preferred. In an embodiment, the FXa inhibitor is orally administered to a subject with cancer, including metastatic cancer, who is in need of treatment. In a particular embodiment, the FXa inhibitor edoxaban p-toluenesulfonate monohydrate is orally administered to a subject with cancer, including metastatic cancer, who is in need of treatment.

The amount of active ingredient that may be combined with a carrier material to produce a single dosage form will vary depending upon the host treated and the particular mode of administration. The precise amount of compound administered to a patient will be the responsibility of the attendant physician and route of administration. The specific dose level for any particular patient will depend upon a variety of factors, including the activity of the specific compound employed, the age, body weight, general health, sex, diet, time of administration, route of administration, rate of excretion, drug combination, the precise disorder being treated, and the severity of the disorder being treated. Also, the route of administration may vary depending on the disorder and its severity.

In an embodiment, the cancers treatable by the methods of the present invention may have been treated by surgical intervention (removal of all or a portion of the cancer), and/or with one or more chemotherapeutic agents, prior to treatment with the Factor Xa inhibitor, such as edoxaban. In addition, the methods can further include treatment with the FXa inhibitor, e.g., edoxaban, in conjunction with one or more additional therapeutic or chemotherapeutic agents. Such chemotherapeutic agents may include, without limitation, alkylating agents, for example, nitrogen mustards, ethyleneimine compounds and alkyl sulfonates; antimetabolites, for example, folic acid, purine or pyrimidine antagonists; mitotic inhibitors, for example, vinca alkaloids and derivatives of podophyllotoxin; cytotoxic antibiotics; and compounds that damage or interfere with DNA expression. In an embodiment, radiation therapy may be administered to the subject in addition to other adjunct or adjuvant treatments.

In some embodiments, the method of the invention involves combining treatment with an inhibitor of Factor Xa, in particular, edoxaban, with another type of cancer treatment, e.g., chemotherapy, radiation therapy, immunotherapy, or surgery. Treatment with the Factor Xa inhibitor, e.g., edoxaban, may occur after another type of cancer treatment; it may be prophylactic; or it may occur at the same time. The treatment may also be directly administered to affect primary or metastatic tumor progression or growth. By way of example, a statistically significant change in the numbers of primary tumor or metastasizing cells will typically be at least about 10%, preferably 20%, 30%, 50%, 70%, 90%, or more. The effects may be specific in blocking tumor growth or progression to specific points.

In other embodiments, the methods of the invention can involve combining treatment of a subject, particularly a subject with a cancer or neoplasm, with an inhibitor of Factor Xa, in particular, edoxaban, with another anticoagulant agent, anti-thrombotic agent, and/or anti-FXa agent as secondary or adjunct treatment. Such secondary or adjunct treatments may be given prior to, concomitantly with, or subsequent to treatment with edoxaban. In some embodiments, the agent is heparin. In other embodiments, the agent may be an oral anticoagulants, including, without limitation, Vitamin K antagonists, (e.g, warfarin, dicumarol, or coumarin derivatives), Factor IIa inhibitors (e.g., dabigatran), and other FXa inhibitors (e.g., rivaroxaban, apixaban). In other embodiments, parenteral anticoagulant agents include, without limitation, heparin, low molecular weight heparins (e.g., dalteparin, tinzaparin, reviparin, nadroparin, ardeparin, certoparin and parnaparin), or direct thrombin inhibitors (e.g., bivalirudin, argatroban, desirudin and lepirudin). Other parenteral FXa inhibitors include fondaparinux.

In its embodiments, the invention provides methods conducive to improving treatments and treatment options for individuals afflicted with cancer, including metastatic and/or malignant cancer, wherein the individuals can particularly benefit from treatment or therapy with edoxaban. In addition, the invention provides improved treatment methods in which individuals afflicted with both cancer and a thrombotic condition or embolism can benefit substantially from treatment with one primary drug, namely, a direct FXa inhibitor, e.g., edoxaban. Thus, the term “treating” in a general sense as used herein refers to preventing, inhibiting, curing, reversing, attenuating, alleviating, abrogating, minimizing, suppressing, reducing, or eliminating the deleterious effects of a disease state, disease progression, disease causative agent, e.g., bacteria or viruses, or other abnormal condition, such as a non-benign cancer, tumor, or neoplasm. For example, treatment may involve alleviating a symptom, although not necessarily all of the symptoms of a disease, or attenuating the progression of a disease. The treatment of cancer, as used herein, refers to partially or totally inhibiting, eliminating, delaying, reversing, reducing, or preventing the progression of cancer, including cancer metastasis or malignancy, and/or the recurrence of cancer, including cancer metastasis or malignancy; or preventing the onset or development of cancer in a mammal, in particular, a human. The treatment of cancer further refers to the reduction of the incidence of migration, invasion, or progression of a metastasis or a malignant cancer, or the reduction of the incidence of mets, particularly in a human subject undergoing treatment with a Factor Xa inhibitor such as edoxaban.

As will be appreciated by the skilled practitioner, the FXa inhibitor, e.g., edoxaban, pursuant to the invention is preferably used in a therapeutically effective amount, which is intended to qualify as the amount or dose of the treatment, e.g., drug, compound, active ingredient, composition, or agent, determined or necessary to treat cancer in a therapeutic or treatment regimen. This includes combination therapy involving the use of multiple therapeutic agents, such as a combined amount of a first and second treatment, in which the combined amount will achieve the desired biological treatment response as described herein above. As is appreciated by those skilled in the art, “therapeutically effective amount” refers to the amount of a drug or compound that, when administered, is sufficient to prevent the development of, or reduce, alleviate, or abrogate to some extent, one or more of the symptoms of the disorder being treated. The term “therapeutically effective amount” also refers to the amount of a drug or compound that is sufficient to elicit a biological or medical response of a cell, tissue, system, animal, or human that is being sought by a practitioner, e.g., a medical doctor, clinician, veterinarian, or researcher.

In cases in which the patient's condition does not improve, upon the doctor's discretion, an FXa inhibitor, e.g., edoxaban, may be chronically administered, that is, for an extended period of time, including a number of months or years, or even throughout the duration of the patient's lifetime, in order to ameliorate or otherwise control or limit the symptoms of the patient's disorder. In cases in which the patient's status does improve, upon the doctor's discretion, the administration of an FXa inhibitor, e.g., edoxaban, may be given acutely, or temporarily suspended for a certain length of time (i.e., a “drug holiday”). Once improvement of the patient's conditions has occurred, a maintenance dose can be administered, if or as necessary. Subsequently, the dosage or the frequency of administration, or both, can be reduced, as a function of the symptoms or effect on the disease or condition, to a level at which improvement of the disease or condition is retained or maintained. As needed, patients can require intermittent or periodic treatment on a long-term basis upon any recurrence of disease symptoms, such as relapse, mets, and the like.

The present invention provides a new and desirable treatment modality for reducing the incidence of malignancies or metastases in human subjects afflicted with, or at risk for, cancer, particularly malignant or metastatic cancer. As provided by the invention, a direct inhibitor of Factor Xa was surprisingly found to significantly reduce the incidence of malignant or metastatic cancers in human patients undergoing treatment with the Factor Xa inhibitor for a thrombotic condition, including but not limited to, deep vein thrombosis, venous thromboemolism, pulmonary embolism, thromboembolism, and venous thrombosis. The Factor Xa inhibitor in accordance with an embodiment of the invention and as described supra is edoxaban. The invention advantageously provides an agent, e.g., edoxaban, that functions both as a direct inhibitor of Factor Xa activity and to prevent or reduce the spread or progression of metastases and malignant cancers. Such an agent, e.g., edoxaban, serves as an effective, beneficial and economical means of treating those individuals who suffer from, or are at risk for, both thrombosis of various types and cancer of various types, including malignant or metastatic cancers.

### EXAMPLES

### Example 1

This Example describes the clinical study and result thereof in which edoxaban treatment was unexpectedly discovered to reduce or suppress the incidence of several types of cancers in patients having cancer and undergoing treatment with the drug edoxaban as compared to warfarin.

The study (Hokusai VTE Clinical Study) constituted a phase 3, randomized, double-blind, double-dummy, parallel-group, multi-center, multi-national study for the evaluation of efficacy and safety of (LMW) heparin/edoxaban versus (LMW) heparin/warfarin in subjects with symptomatic deep-vein thrombosis and/or pulmonary embolism. The study indication related to the reduction of the risk of symptomatic recurrent venous thromboembolic complications in patients with acute symptomatic deep vein thrombosis (DVT) and/or pulmonary embolism (PE). The primary objective of the study was to evaluate whether initial (LMW) heparin followed by edoxaban only ([LMW] heparin/edoxaban) was non-inferior to initial (LMW) heparin overlapping with warfarin, followed by warfarin only ([LMW] heparin/warfarin) in the treatment of subjects with acute symptomatic VTE for the prevention of symptomatic recurrent VTE during the 12-month study period. Accordingly, the nature of the study was the use of edoxaban as direct inhibitor of Factor Xa in the treatment of VTE.

Secondary goals of the study involved a comparison of (LMW) heparin/edoxaban to (LMW) heparin/warfarin with regard to clinically relevant bleeding (i.e., major or clinically relevant non-major bleeding) occurring during treatment or within 3 days of interrupting or stopping study drug and with regard to the composite clinical outcome of symptomatic recurrent DVT, non-fatal symptomatic recurrent PE, and all-cause mortality during the 12-month study period.

Dose Selection

The recurrence of VTE is highest within the first 3 months—estimated at 2.5%—following the index event. Subsequently, the event rate is lower but remains constant thereafter—at 0.5% per quarter. Thus, the incidence of unwanted bleeding due to anticoagulant therapy is highest in the first 1-2 weeks and then remains constant as long as therapy is administered. Hence, the ratio between the risk of recurrent thromboembolism and the risk of bleeding changes over time as it also changes the treatment benefit/risk. Previous studies have suggested that the inflection point occurs at around 3 months after the initial diagnosis and therapeutic intervention (Fiessinger et al., JAMA, 2005, 293: 681 and Buller, et al., NEJM, 2007, 357: 1094). Based on dose selection decisions made in previous programs, this study adopted an edoxaban dosing regimen of 60-mg QD, in which edoxaban was administered as the p-toluenesulfonate salt form and the dose was calculated as the free base form. This regimen was selected to provide optimal anticoagulant effect, with a bleeding risk projected to be no worse than, and possibly somewhat better than, that associated with warfarin use.

Population

The study included all patients with a presumptive diagnosis of VTE based on standard diagnostic procedures at the study site. All index cases were ultimately verified by independent and blinded adjudication subsequent to randomization. Following the site-diagnosis of VTE, but prior to randomization, subjects were stratified by 1) presenting diagnosis ((a) PE with or without DVT and (b) DVT only), 2) risk characterization and 3) need for dose adjustment (body weight <60 kg; creatinine clearance between 30 and 50 mL/min, and concomitant use of strong P-glycoprotein inhibitors). Risk characterization, patterned after ACCP Guidelines, dichotomized patients into those with a) transient, provoked risk factors (e.g., major surgery, trauma, immobilization) and b) all others. The study sought to enroll at least 40% of subjects presenting with PE (with or without DVT) and to cap the proportion of subjects with transient, provoked risk factors at 10%.

Treatment Regimens

Following stratification and treatment allocation as described above, all patients were randomized to either a standard of care (SOC) treatment regimen, i.e., heparin+warfarin, until the international normalized ratio (INR) was between 2-3, followed by warfarin alone; or an edoxaban treatment regimen, i.e., heparin for 5 days followed by edoxaban (as the free base) at a dose of 60-mg QD (or 30-mg QD, in the low exposure group). This study aimed to treat all patients for the entire 12 month period following randomization. Ultimately, the evolving nature of the patient's clinical status and changes in the risk/benefit over time determined the length of treatment for a given patient. Irrespective of actual treatment duration, all patients were followed for the entire 12 months after randomization.

Hypotheses

The study tested whether treatment with enoxaparin/edoxaban was non-inferior to treatment with enoxaparin/warfarin in preventing recurrence of acute, symptomatic VTE following an initial index event. Assuming a total of 256 events across both treatment groups, enoxaparin/edoxaban would be considered non-inferior to the standard therapy if the upper limit of the two-sided 95% confidence interval for the risk ratio (Edoxaban/Standard Therapy) were less than 1.5. This corresponds to preservation of at least 70% of the minimal warfarin effect.

Primary and Secondary Objectives

The primary efficacy objective was to demonstrate non-inferiority of enoxaparin/edoxaban to enoxaparin/warfarin in the treatment of subjects with acute symptomatic VTE (PE with or without DVT or DVT only) and the prevention of recurrent venous thromboembolic events during the 12-month study period. If non-inferiority were established, enoxaparin/edoxaban would be compared to enoxaparin/warfarin for superiority. Secondary objectives included (i) a comparison of enoxaparin/edoxaban to enoxaparan/warfarin with regard to the composite clinical outcome of recurrent DVT, non-fatal recurrent PE, and all-cause mortality, as well as each component separately during the 12-month study period; and (ii) a comparison of enoxaparin/edoxaban to enoxaparin/warfarin with regard to major and clinically relevant non-major bleeding, as well as each component separately occurring during the on-treatment study period.

Study Population

Adult patients presenting with acute, symptomatic deep venous thrombosis involving the popliteal, femoral or ileac veins, or pulmonary embolism requiring anti-thrombotic therapy are eligible for study. Diagnostic criteria for DVT were: 1) a noncompressible vein on ultrasonography, or 2) an intraluminal filling defect on venography. Diagnostic criteria for PE were: 1) an intraluminal filling defect on spiral CT or pulmonary angiography, 2) cutoff of contrast material in a vessel more than 2.5 mm in diameter on pulmonary angiography, 3) a perfusion defect involving at least 75 percent of a segment, with corresponding normal ventilation, or 4) a non-diagnostic lung scan accompanied by documentation of new deep vein thrombosis by ultrasonography or venography.

Dosage Forms, Formulations and Routes of Administration

Edoxaban was administered in 30 and 60 mg dose-strengths in tablet form. Warfarin was administered in 1, 2.5 and 5 mg dose-strengths. Matching placebos were used for each of the test drugs. Edoxaban and warfarin were administered orally (PO). Enoxaparin was administered by subcutaneous (SC) injection.

Study Design and Treatment Duration

The study was an event-driven, Phase 3, multi-national, multi-center, randomized, double-blind, matching placebo, parallel-group non-inferiority study for efficacy. The total study period after randomization was 12 months in duration. Ideally, all subjects remained on study treatment throughout this period. However, mitigating factors related to the subject's clinical status could influence the total duration of treatment a given subject actually received (See, Treatment Arms, below). Nevertheless, all subjects were administered a minimum of three months' treatment consistent with current ACCP Guidelines. Regardless of the total duration of treatment actually received, efficacy and safety data were collected for all subjects, including those who temporarily interrupted or permanently discontinued study drug, at routine clinic visits during the entire 12-month study period following randomization. In addition to the final month 12 study visit, all randomized subjects who completed an entire 12 months of study treatment were contacted by phone 30 days after discontinuing treatment to collect adverse event (AE) data.

Treatment Arms

Eligible subjects were stratified by 1) presenting diagnosis ((a) PE with or without DVT and (b) DVT only); 2) risk characterization; and 3) need for dose adjustment (body weight <60 kg; creatinine clearance between 30 and 50 mL/min, and concomitant use of strong P-glycoprotein (P-gp) inhibitors). Risk characterization, patterned after ACCP Guidelines, dichotomized patients into those with a) transient, provoked risk factors (e.g., major surgery, trauma, immobilization) and b) all others. Anti-coagulation treatment, including up to a single dose of VKA was allowed for a maximum of 36 hours prior to randomization. Upon confirmation of eligibility, subjects were assigned randomly via interactive voice response system (IVRS) in a 1:1 ratio to one of two treatment groups: the enoxaparin/edoxaban group or the enoxaparin/warfarin group.

Enoxaparan/Edoxaban Group: Enoxaparin, 1 mg/kg bid, plus placebo warfarin, were administered for the initial 5 days of treatment. Starting on Day 6, subjects were administered placebo enoxaparin, edoxaban 60 mg QD and placebo warfarin. To protect the blind, administration of placebo enoxaparin was continued until the sham INR reached 2:2 on two consecutive readings at least 24 hours apart. Afterward, subjects continued on edoxaban 60 mg plus placebo warfarin QD for the remainder of dosing.

Enoxaparan/Warfarin Group: Enoxaparin, 1 mg/kg bid, plus warfarin, were administered for the initial 5 days of treatment. Starting on Day 6, placebo edoxaban QD was added to the enoxaparin/warfarin regimen. Enoxaparin administration continued until the INR reached 2:2 on two consecutive readings at least 24 hours apart. Afterward, subjects continued on warfarin and placebo edoxaban for the remainder of dosing.

Dose Adjustment

In the edoxaban group, the dosage was halved to 30 mg QD for subjects with moderate renal impairment (CrCl 30-50 mL/min), body weight 60 kg, or concurrently receiving a strong P-gp inhibitor, such as quinidine or verapamil. For subjects who had their edoxaban dose adjusted at the time of randomization, the edoxaban dosage regimen was reduced permanently, even if the subject subsequently gained weight, experienced improved CrCl, or discontinued treatment with P-gp inhibitors. After randomization, if the subject's body weight dropped to 60 kg (confirmed by repeat measurement within one week) and the body weight change was >10% of the subject's baseline body weight, the edoxaban dosage regimen was reduced permanently, even if the subject subsequently regained weight to >60 kg. After randomization, if the subject's CrCl became ≦50 mL/min and ≧30 mL/min (confirmed by repeat measurement within one week) and the CrCl change was >10% of the subject's baseline CrCl, the Edoxaban dosage regimen was reduced permanently, even if the subject subsequently experienced improved CrCl to >50 mL/min. In the warfarin group, INR was periodically monitored and warfarin dosage adjusted accordingly to maintain a therapeutic INR between 2.0 and 3.0.

Statistical Analysis

The primary study analysis compared treatment efficacy for the first occurrence of a primary efficacy endpoint event (DVT, non-fatal and fatal PE) for all subjects in the modified Intent-to-Treat (mITT) subjects (all subjects who were randomized, received at least one dose of study drug and had an adjudicated/confirmed index (baseline) VTE event) analysis set using an on-treatment method. Events were counted in this analysis only if they occurred while the subject was “at risk” while on study drug or within 3 days following the last dose. The time to the first event of the composite primary efficacy outcome was analyzed using a stratified Cox's proportional hazard model. The time to first event was defined as the time “at risk” from the day of initial study drug dose to the first event experienced by a subject. For this analysis, patients who did not have a VTE event while “at risk” during the 12 month study period, or patients lost to follow-up, or patients who died because of other reasons than DVT/PE, or patients who withdrew informed consent before the end of the 12 month study period, were censored at the last day the patient had a complete assessment for study outcomes, 3 days after final dose or death due to reasons unrelated to the DVT/PE, whichever came first. The enoxaparin/edoxaban-to-comparator hazard ratio was estimated with a 95% CI, based on this model. Enoxaparin/edoxaban was considered non-inferiority to the comparator if the upper limit of the 95% CI was less than 1.5.

A sensitivity analysis was performed for the mITT analysis set. This analysis included all events that occurred during the 12 month study period. The aforementioned statistical method described for the primary efficacy analysis was applied. Subjects were also categorized into two groups based upon baseline VTE status. One group consisted of subjects with DVT without PE and the other group consisted of subjects with PE with/without DVT. Analysis of VTE event rate was performed for each group for the mITT analysis set with the on-treatment approach. The event rate of VTE was estimated and a 95% confidence interval for the hazard ratio was constructed for each group.

Superiority of enoxaparin/edoxaban versus enoxaparin/warfarin was tested if non-inferiority was first established for enoxaparin/edoxaban. The superiority analysis was performed for the ITT analysis set (including all randomized subjects) with all events in the 12 month study period counted, including events occurring while off study drug. The time to first event was estimated by Kaplan-Meier estimate and was compared between the two treatment groups using a log-rank test, at a nominal significance level of α=0.01. Secondary efficacy endpoint was analyzed based on the ITT analysis set. Superiority of enoxaparin/edoxaban as compared to enoxaparin/warfarin for the secondary efficacy endpoint (composite clinical outcome of recurrent DVT, non-fatal recurrent PE, and all-cause mortality) was tested only if superiority was first established for enoxaparin/edoxaban with respect to the primary efficacy outcome. The secondary efficacy endpoint was analyzed based on the ITT analysis set.

The time to first event of the secondary efficacy endpoint (composite clinical outcome of recurrent DVT, non-fatal recurrent PE, and all-cause mortality as well as each component separately) was analyzed using the same statistical method (and α=0.01) as utilized in the superiority analysis of the primary efficacy endpoint. If superiority of enoxaparin/edoxaban versus enoxaparin/warfarin in the composite clinical endpoint of recurrent DVT, non-fatal recurrent PE, and all-cause mortality was established, enoxaparin/edoxaban was compared with enoxaparin/warfarin for superiority for major and clinically relevant non-major bleeding using the on-treatment approach. The time to the first occurrence of major and clinically relevant non-major bleeding was compared between the treatment groups, using the same statistical method (and α=0.01) as in the superiority analysis of the primary efficacy endpoint. If the test was statistically significant, time to major bleeding only was also tested at the same significance level using the same method as for the major and clinically non-major bleeding events.

Analysis of Subjects with Cancer Who were Treated with Edoxaban in the VTE Study

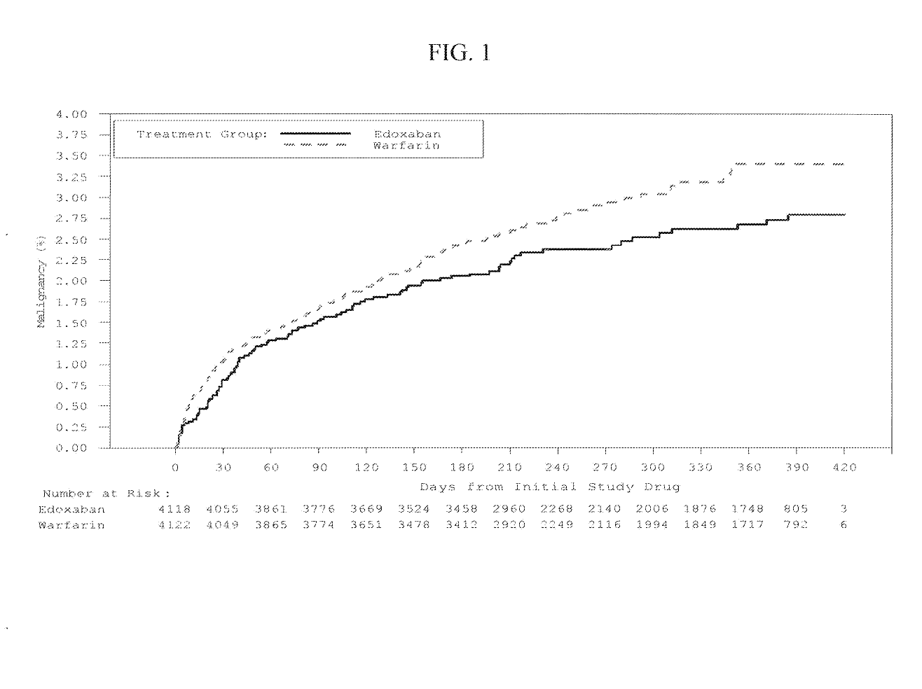

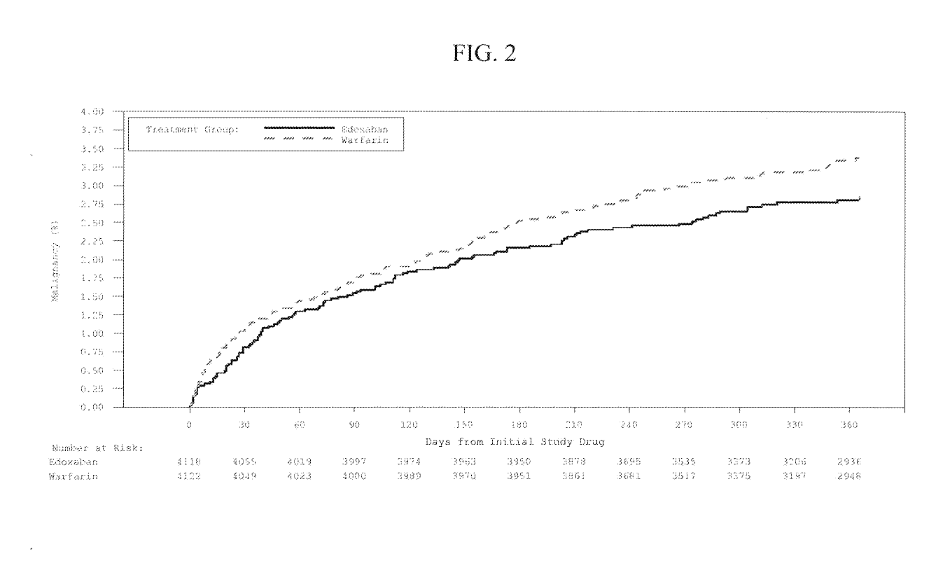

Subject data from the clinical study were prospectively analyzed to gain safety information regarding the potential for incidence of new cancers in the edoxaban study program. The study included subject populations who had cancer upon entering the study. Upon examination of the Hazard Ratios (HRs) in different population sets, e.g., “Safety Overall” and “Safety on Treatment plus (+) 30 Days”, as well as the respective Kaplan-Meier plots (See, FIGS. 1 and 2), a positive trend was observed for the edoxaban treated patients. The HRs were nearly statistically significant, with a reduction in the incidence of investigator reported malignancies clinically evident post-randomization ranging from 16% to 27%. (See, e.g., Tables 1-3). The cancers affected (reduced or suppressed) by edoxaban treatment were predominantly, but not exclusively, solid. The findings based on cancer patient data analysis from the edoxaban clinical study program revealed that administration of edoxaban resulted in a reduced incidence of new or recurrent malignancies, and thus, the ability of a direct Factor Xa inhibitor, such as edoxaban, to prevent, suppress, treat, or reduce cancer and/or metastatic spread or progression in patients suffering from cancers and undergoing treatment with the Factor Xa inhibitor, such as edoxaban.

The results of the analysis of the patients having cancer and treated with edoxaban in the VTE study are provided in the following tables. Table 1 presents a summary of the investigator-reported malignancies that were clinically evident following randomization of subjects as evaluated at the time of “On-Treatment Study Period”, i.e., the period of time during which study drugs were administered to subjects in the study, in the Hokusai VTE clinical study described herein.

TABLE 1

| | | Edoxaban | | Warfarin |
|---|---|---|---|---|
| | Statistic | (N = 4118) | | (N = 4122) |
| All Subjects with a | n (%) | 66 | (1.6) | 90 (2.2) |
| Malignancy Event | HR | 0.73 | (0.531, 1.003) | |
| | (95% CI) | | | |
| Cancer/Tumor Type - | | | | |
| Solid Tumor | n (%) | 61 | (1.5) | 84 (2.0) |
| Leukemia, | n (%) | 4 | (<0.1) | 6 (0.1) |
| Lymphoma, and | | | | |
| Other Blood | | | | |
| Malignancies | | | | |
| Diagnosed as a | | | | |
| result of work-up | | | | |
| for anemia or | | | | |
| bleeding | | | | |
| Yes | n (%) | 9 | (0.2) | 10 (0.2) |
| No | n (%) | 57 | (1.4) | 80 (1.9) |

In Table 1, “N” refers to the number of subjects assessed. The study data are presented as n (%), which indicates the number (n) of patients found to have a new case of cancer; the number in parenthesis indicates the incidence of a cancer expressed as a percentage (%) of the population randomized to the designated treatment group; “HR” refers to Hazard Ratio, which describes the relative risk of a complication based on a comparison of event rates; and “CI” refers to Confidence Interval. Events are included in the On-Treatment Study Period if they occurred on or after the date of first dose of any study drug. Events that start after the third day following the calendar date of any ‘last dose’ and before the date of the next ‘first dose’ are not considered for the On-Treatment Study Period. The summary includes all reported malignancy events, which were diagnosed after randomization. The HR and two-sided Clare based on the Cox proportional hazards regression model including treatment and the following randomization stratification factors as covariates: presenting diagnosis (PE with or without DVT; DVT only), baseline risk factors (temporary factors; all others), and the need for dose reduction (yes; no). Table 1 demonstrates the finding that fewer edoxaban-treated patients had malignancy events than patients treated with warfarin. The edoxaban-treated patients showed fewer, i.e., a reduction in, solid tumors and blood-related cancers than did the patients treated with warfarin. Similar results were observed upon analysis of cancer patient data at the other time points in the study, as presented in Tables 2 and 3 below.

Table 2 presents a summary of the investigator-reported malignancies that were clinically evident post-randomization as evaluated at the time of “Treatment+30 Days Study Period” in the Hokusai VTE clinical study described herein. The “Treatment+30 Days Study Period” refers to the overall period of study drug administration, study drug interruption and 30-day period (for post-study safety assessment).

TABLE 2

| | | Edoxaban | | Warfarin |
|---|---|---|---|---|
| | Statistic | (N = 4118) | | (N = 4122) |
| All Subjects with a | n (%) | 99 | (2.4) | 118 (2.9) |
| Malignancy Event | HR | 0.84 | (0.640, 1.091) | |
| | (95% CI) | | | |
| Cancer/Tumor Type - | | | | |
| Solid Tumor | n (%) | 93 | (2.3) | 109 (2.6) |
| Leukemia, | n (%) | 6 | (0.1) | 9 (0.2) |
| Lymphoma, and | | | | |
| Other Blood | | | | |
| Malignancies | | | | |
| Diagnosed as a | | | | |
| result of work-up | | | | |
| for anemia or | | | | |
| bleeding | | | | |
| Yes | n (%) | 23 | (0.6) | 20 (0.5) |
| No | n (%) | 76 | (1.8) | 98 (2.4) |

In Table 2, “N”, “n (%)”, “HR” and “CI” are as described for Table 1. Events are included in the Treatment+30 Days Study Period if they occurred on or after the date of first dose of any study drug. Events that start after thirty days following the last dose of study drug are not considered for the Treatment+30 Days Study Period. The summary includes all reported malignancy events which were diagnosed after randomization. The HR and two-sided Clare based on the Cox proportional hazards regression model including treatment and the following randomization stratification factors as covariates: presenting diagnosis (PE with or without DVT; DVT only), baseline risk factors (temporary factors; all others), and the need for dose reduction (yes; no).

Table 3 presents a summary of the investigator-reported malignancies that were clinically evident post-randomization as evaluated at the time of “Overall Study Period”, i.e., the overall period of study drug administration, study drug interruption and post-treatment up to 12 months (365 days), in the Hokusai VTE clinical study described herein.

TABLE 3

| | | Edoxaban | | Warfarin |
|---|---|---|---|---|
| | Statistic | (N = 4118) | | (N = 4122) |
| All Subjects with a | n (%) | 117 | (2.8) | 137 (3.3) |
| Malignancy Event | HR | 0.85 | (0.666, 1.091) | |
| | (95% CI) | | | |
| Type - | | | | |
| Solid Tumor | n (%) | 110 | (2.7) | 127 (3.1) |
| Leukemia, | n (%) | 7 | (0.2) | 10 (0.2) |
| Lymphoma, and | | | | |
| Other Blood | | | | |
| Malignancies | | | | |
| Diagnosed as | | | | |
| a result of work-up | | | | |
| for anemia or | | | | |
| bleeding | | | | |
| Yes | n (%) | 25 | (0.6) | 22 (0.5) |
| No | n (%) | 92 | (2.2) | 115 (2.8) |

In Table 3, “N”, “n (%)”, “HR” and “CI” are as described for Tables 1 and 2. Events are included in the overall study period if they occurred on or after the date of first dose of any study drug. All events that start prior to last study follow-up contact are considered for the Overall Study Period. The summary includes all reported malignancy events which were diagnosed after randomization. The HR and two-sided Clare based on the Cox proportional hazards regression model as described supra for Tables 1 and 2.

It is to be understood that suitable methods and materials are described herein for the practice of the embodiments; however, methods and materials that are similar or equivalent to those described herein can be used in the practice or testing of the invention and described embodiments.

All publications, patent applications, patents, and other references mentioned herein are incorporated by reference in their entireties. [Edit as necessary] Certain modifications and improvements will occur to those skilled in the art upon a reading of the foregoing description. It should be understood that all such modifications and improvements have been deleted herein for the sake of conciseness and readability but are properly within the scope of the following claims.

1. A method of treating or preventing a cancer or neoplasm in a human subject in need thereof, said method comprising administering to the subject a pharmaceutical composition comprising a direct Factor Xa inhibitor in a therapeutically effective amount to said human subject.

2. The method according to claim 1 wherein the direct Factor Xa inhibitor is N¹-(5-chloropyridin-2-yl)-N²-((1 S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, having the structure: 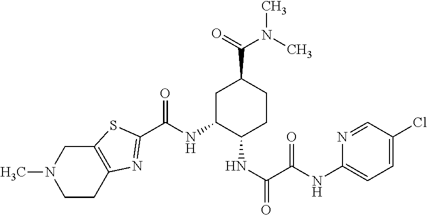 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]
or a pharmaceutically acceptable salt and/or hydrate thereof.

3. The method according to claim 1 wherein the direct Factor Xa inhibitor is edoxaban p-toluenesulfonate monohydrate, having the formula: N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide p-toluenesulfonate monohydrate, and the structure:

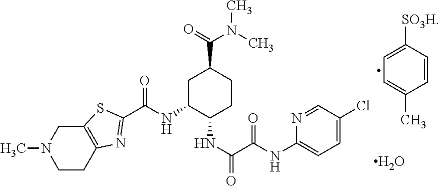

4. The method according to claim 3, wherein edoxaban is in a free base form or equivalent thereof.

5. The method according to claim 4, wherein the effective amount of edoxaban is up to 90 mg per day.

6. The method according to claim 4, wherein the effective amount of edoxaban is 60 mg per day.

7. The method according to claim 4, wherein the effective amount of edoxaban is 30 mg per day.

8. The method according to claim 4, wherein the administering comprises oral administration.

9. The method according to claim 4, wherein the direct Factor Xa inhibitor is in a solid oral form.

10. The method according to claim 1, wherein the cancer or neoplasm is selected from a solid tumor, a solid neoplasm, a non-solid tumor, a non-solid neoplasm, a lymphoma, or a leukemia.

11. The method according to claim 10, wherein the cancer or neoplasm is located in or on a body tissue or organ selected from thyroid, lung, stomach, small bowel, large bowel, liver, kidney, pancreas, prostate, uterus, genital, or bladder.

12. The method according to claim 1, wherein the FXa inhibitor effectively functions (i) as an antithrombotic and anticoagulant agent and (ii) as an inhibitor of incidence of metastasis or tumor migration.

13. The method according to claim 1, further comprising treating the subject with an additional chemotherapeutic agent.

14. The method according to claim 13, wherein the chemotherapeutic agent is selected from the group consisting of an alkylating agent, an anti-metabolite, a mitotic inhibitor, a cytotoxic antibiotic, a compound that damages DNA and a compound that interferes with DNA expression.

15. The method according to claim 1, further comprising treating the subject with surgery and/or radiotherapy.

16. A method of treating a cancer or neoplasm in a human subject, comprising:
administering to the subject a pharmacologically effective amount of heparin;
subsequently discontinuing heparin administration to the subject; and
administering a pharmaceutical composition comprising a direct Factor Xa inhibitor in a pharmacologically effective amount to the subject, thereby treating the cancer or neoplasm in the subject.

17. The method according to claim 16, wherein administration of heparin is discontinued after about five days.

18. A method of reducing the incidence of metastasis of a cancer or neoplasm in a human subject in need thereof, the method comprising administering to the subject an effective amount of a direct Factor Xa inhibitor.

19. (canceled)

20. The method according to claim 16 or 18, wherein the direct Factor Xa inhibitor is N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, the Factor Xa inhibitor having the structure: 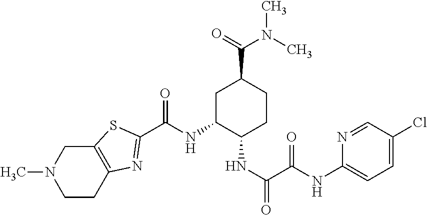 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]
or a pharmaceutically acceptable salt and/or hydrate thereof.

21. The method according to claim 16 or 18, wherein the direct Factor Xa inhibitor is edoxaban p-toluenesulfonate monohydrate, having the formula: N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide p-toluenesulfonate monohydrate, and the structure:

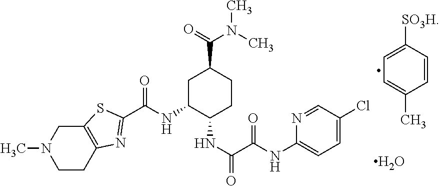

22. A method of reducing the formation or progression of a cancer or neoplasm in a human subject need thereof and having thrombotic disease, the method comprising treating the subject with an effective amount of a direct Factor Xa inhibitor having the formula: N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, and the structure: 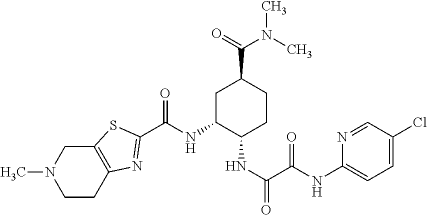 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]
or a pharmaceutically acceptable salt and/or hydrate thereof.

23. The method according to claim 22, wherein the direct Factor Xa inhibitor is edoxaban p-toluenesulfonate monohydrate having the formula: N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide p-toluenesulfonate monohydrate, and the structure:

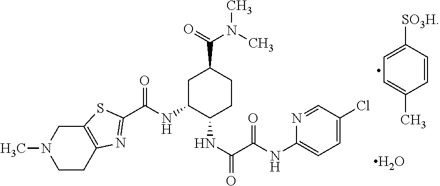

24. The method according to claim 23, wherein edoxaban is in a free base form or equivalent thereof.

25. The method according to claim 24, wherein the effective amount of edoxaban is up to 90 mg per day.

26. The method according to claim 24, wherein the effective amount of edoxaban is 60 mg per day.

27. The method according to claim 24, wherein the effective amount of edoxaban is 30 mg per day.

28. The method according to claim 1, wherein the administering comprises oral administration.

29. The method according to any claim 1, wherein the direct Factor Xa inhibitor is in a solid oral form.

30. The method according to claim 1, wherein the cancer or neoplasm is selected from a solid tumor, a solid neoplasm, a non-solid tumor, a non-solid neoplasm, a lymphoma, or a leukemia.

31. The method according to claim 30, wherein the cancer or neoplasm is located in or on a body tissue or organ selected from thyroid, lung, stomach, small bowel, large bowel, liver, kidney, pancreas, prostate, uterus, genital, or bladder.

32. (canceled)

33. The method according to claim 1, further comprising treating the subject with an additional chemotherapeutic agent.

34. The method according to claim 33, wherein the chemotherapeutic agent is selected from the group consisting of an alkylating agent, an anti-metabolite, a mitotic inhibitor, a cytotoxic antibiotic, a compound that damages DNA and a compound that interferes with DNA expression.

35. The method according to claim 1, further comprising treating the subject with surgery and/or radiotherapy.

36. The method according to claim 1, wherein the human subject suffers from a condition selected from venous thromboembolism, deep vein thrombosis pulmonary embolism, embolism, thromboembolism, and venous thrombosis, cerebral infarction, cerebral embolism, myocardial infarction, angina pectoris, pulmonary infarction, pulmonary embolism, Buerger's disease, deep venous thrombosis, disseminated intravascular coagulation syndrome, thrombus formation after valve or joint replacement, thrombus formation and reocclusion after angioplasty, systemic inflammatory response syndrome (SIRS), multiple organ dysfunction syndrome (MODS), thrombus formation during extracorporeal circulation, or blood clotting and suffers from, or is at risk for, a cancer, tumor, or neoplasm.

37. The method according to claim 1, wherein the human subject is an individual who currently has cancer: who is in remission from cancer, who is undergoing treatment for a cancer, who may be at risk for developing a cancer, who has a recurrent cancer condition; who has a tumor or neoplasm in which metastasis has not yet occurred; who has a cancer and is in need of anti-thrombotic or anticoagulant treatment; who is in need of anti-thrombotic treatment and who is also at risk for, or has, cancer; or who is in need of an anti-cancer treatment and is also at risk for, or has, thrombotic disease.

38. The method according to claim 37, wherein the cancer comprises a tumor, a neoplasm, a metastatic tumor, a metastatic neoplasm, a malignant tumor, or a malignant neoplasm.

39. The method according to claim 18, wherein the human subject has, or is at risk for, thrombotic disease.
